(19) United States
(12) Patent Application Publication
(10) Pub. No.: US 2026/0138527 A1
(43) Pub. Date: May 21, 2026

(54) INFORMATION PROVISION SYSTEM, INFORMATION PROVISION METHOD, AND STORAGE MEDIUM

(71) Applicant: HONDA MOTOR CO., LTD., Tokyo (JP)

(72) Inventors: Yuichiro Shimura, Wako-shi (JP); Toshihiro Hashimoto, Wako-shi (JP); Haruko Okuyama, Wako-shi (JP); Tsuyoshi Nojiri, Wako-shi (JP); Tatsuya Iwasa, Wako-shi (JP)

(21) Appl. No.: 19/377,141

(22) Filed: Nov. 3, 2025

(30) Foreign Application Priority Data
Nov. 15, 2024 (JP) 2024-199828

(51) Int. Cl. B60Q 9/00; A61B 5/16; A61B 5/18; G06V 20/58; G06V 20/59; G06V 40/16; G10L 13/033; G10L 25/63

(52) U.S. Cl. CPC B60Q 9/00; A61B 5/165; A61B 5/18; G06V 20/58; G06V 20/597; G06V 40/174; G10L 13/033; G10L 25/63

(57) ABSTRACT

An information provision system includes: a risk recognition unit that recognizes a predetermined risk faced by a mobile body; and an agent control unit. The agent control unit: sets an emotion of the agent according to an emotional level associated with happiness and sadness; and when the emotion of the agent is set to a first emotional level, modifies the emotion of the agent to a second emotional level having a degree of happiness lower than that of the first emotional level and outputs warning speech via the agent, when switching from a state in which the predetermined risk is not recognized by the risk recognition unit to a state in which the predetermined risk is recognized by the risk recognition unit.

### INCORPORATION BY REFERENCE

The present application claims priority under 35 U.S.C. § 119 to Japanese Patent Application No. 2024-199828 filed on Nov. 15, 2024. The content of the application is incorporated herein by reference in its entirety.

### BACKGROUND OF THE INVENTION

### Field of the Invention

The present invention relates to an information provision system, an information provision method, and a storage medium.

### Description of the Related Art

In recent years, technology has been proposed to equip a human machine interface (HMI) of an information provision apparatus used in a mobile body such as a vehicle with an anthropomorphic agent that communicates with a user. For example, Japanese Patent Laid-Open No. 2022-038423 discloses a technique of estimating an emotion of a driver of a vehicle, and when the emotion of the driver emotion is negative, i.e., not desiring a response from others, response content is output that can change the emotion of the driver to a negative emotion at a predetermined level or lower or to a positive emotion according to a level of the negative emotion.

When issuing a warning for avoiding contact between the vehicle and an obstacle or the like, through speech output by an HMI agent via a driving assistance function included in the vehicle, it is conceivable to change a mode of the speech output by the agent to enhance effectiveness of the warning. However, due to individual differences in human sensitivity, a driver may be made to feel uneasy depending on how the speech output mode of the agent is changed.

Therefore, an object of the present application is to provide an information provision system that reduces causing a user to feel uneasy, when issuing a warning via the HMI using the agent to the user riding the vehicle. The above technique contributes to further improving traffic safety and development of a sustainable transportation system.

### SUMMARY OF THE INVENTION

As a first aspect to achieve the above object, an information provision system includes: a risk recognition unit that recognizes a predetermined risk faced by a mobile body; and an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.

The above information provision system may further include a user state recognition unit that recognizes a state of the user; and a user emotion estimation unit that estimates an emotion of the user, based on a recognition result of the state of the user by the user state recognition unit, wherein the agent control unit may set the emotion of the agent based on the emotion of the user estimated by the user emotion estimation unit, when the predetermined risk is not recognized by the risk recognition unit.

In the above information provision system, the agent control unit may set the emotion of the agent to an emotional level having a higher degree of happiness than that of an emotional level of the user estimated by the user emotion estimation unit, when the predetermined risk is not recognized by the risk recognition unit.

In the above information provision system, the user emotion estimation unit may use valence as an estimation element of the emotion of the user, and the agent control unit may use valence as a control element of the emotion of the agent.

In the above information provision system, the user emotion estimation unit may use arousal as an estimation element of the emotion of the user, and the agent control unit may use arousal as a control element of the emotion of the agent.

As a second aspect to achieve the above object, an information provision method executed by a computer includes: a risk recognition step of recognizing a predetermined risk faced by a mobile body; and an agent control step of providing, using an agent having an anthropomorphic emotion, a user in the mobile body with a human machine interface (HMI) that performs speech output in a mode corresponding to the emotion of the agent, and outputting warning speech via the agent when the predetermined risk is recognized in the risk recognition step, wherein in the agent control step: the emotion of the agent is set according to an emotional level associated with happiness and sadness; and when the emotion of the agent is set to a first emotional level, the emotion of the agent is modified to a second emotional level having a degree of happiness lower than that of the first emotional level and the warning speech is output via the agent, when switching from a state in which the predetermined risk is not recognized in the risk recognition step to a state in which the predetermined risk is recognized in the risk recognition step.

As a third aspect to achieve the above object, a non-transitory computer-readable storage medium storing a program causes a computer to function as: a risk recognition unit that recognizes a predetermined risk faced by a mobile body; and an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.

According to the information provision system, the information provision method, and the storage medium described above, it is possible to reduce causing a user to feel uneasy, when issuing a warning via the HMI using the agent to the user in the mobile body.

### BRIEF DESCRIPTION OF THE DRAWINGS

FIG. 1 is an explanatory diagram of a use mode of an information provision system;

FIG. 2 is a configuration diagram the information provision system;

FIG. 3 is an explanatory diagram of an emotion map based on valence and arousal;

FIG. 4 is a flowchart of emotion control processing of an agent;

FIG. 5 is an explanatory diagram of an emotional level of the agent with reference to an emotional level of a user;

FIG. 6 is a settings table of the emotional level of the agent with reference to the emotional level of the user;

FIG. 7 shows evaluation data of a difference in receptivity of the user when the emotional level of the agent is changed according to an emotion of the user; and

FIG. 8 shows evaluation data of the difference in the receptivity of the user when the emotional level of the agent is changed to be 1 lower and the user is warned.

### DETAILED DESCRIPTION OF THE INVENTION

### 1. Use Mode of Information Provision System

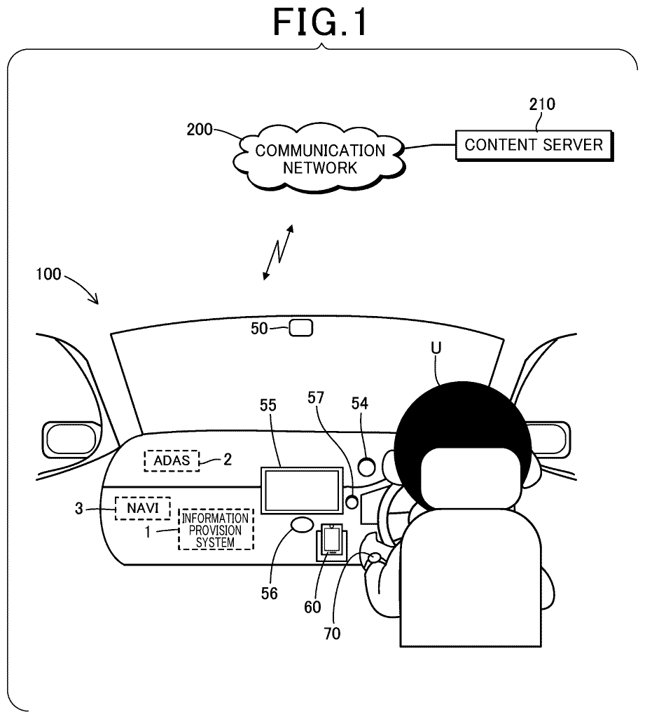

A use mode of an information provision system 1 according to the present embodiment will be described with reference to FIG. 1. The information provision system 1 provides a user U being a driver of a vehicle 100 with a human machine interface (HMI) using an agent with anthropomorphic emotions. The information provision system 1 may be configured as part of functions of an in-vehicle device such as a navigation apparatus installed in the vehicle 100, or may be configured as a dedicated in-vehicle device. The vehicle 100 corresponds to a mobile body of the present disclosure.

The information provision system 1 has a communication function, and performs wireless communication between a wearable device 70 (smartwatch or the like) comprised by a computer and worn by the user U and a mobile terminal 60 (smartphone, tablet terminal, mobile phone, or the like) brought into the vehicle 100 by the user U. The information provision system 1 performs communication with a vehicle-exterior communication system, such as a content server 210, via a communication network 200.

The vehicle 100 includes a driver monitor camera 54 that captures the user U, a touch panel display 55, a speaker 56, a microphone 57 that collects an utterance of the user U, a front camera 50 that captures an area in front of the vehicle 100, an advanced driver-assistance system (ADAS) 2 that performs various driving assistance control based on an image captured by the front camera 50, and a navigation apparatus 3 that performs route guidance to a destination.

The agent installed in the information provision system 1 is, for example, an interface agent including an artificial intelligence (AI) program. The agent is an interactive interface that analyzes the speech of the user U input to the microphone 57 through speech recognition, and outputs information through speech output from the speaker 56 or through image display on the display 55 according to utterance content. The agent, depending on an utterance of the user U, suggests a destination via the route guidance of the navigation apparatus 3, suggests content to be downloaded from the content server 210 and played, and provides a warning and the like about a predetermined risk (approaching obstacle or the like) recognized by the ADAS 2.

The information provision system 1 estimates an emotion of the user U based on the image of the user U captured by the driver monitor camera 54, the speech of the user U input to the microphone 57, and the biological information (heart rate, body temperature, blood pressure, and the like) of the user U detected by the wearable device 70.

The information provision system 1 sets the emotion of the agent based on the emotion of the user U, and the agent controls a mode of the speech output from the speaker 56 (utterance content, intonation, pitch, sound volume, and the like) according to the set emotion. As will be described in detail below, the information provision system 1 changes the emotional level of the agent and outputs warning speech, when the predetermined risk is recognized.

### 2. Configuration of Information Provision System

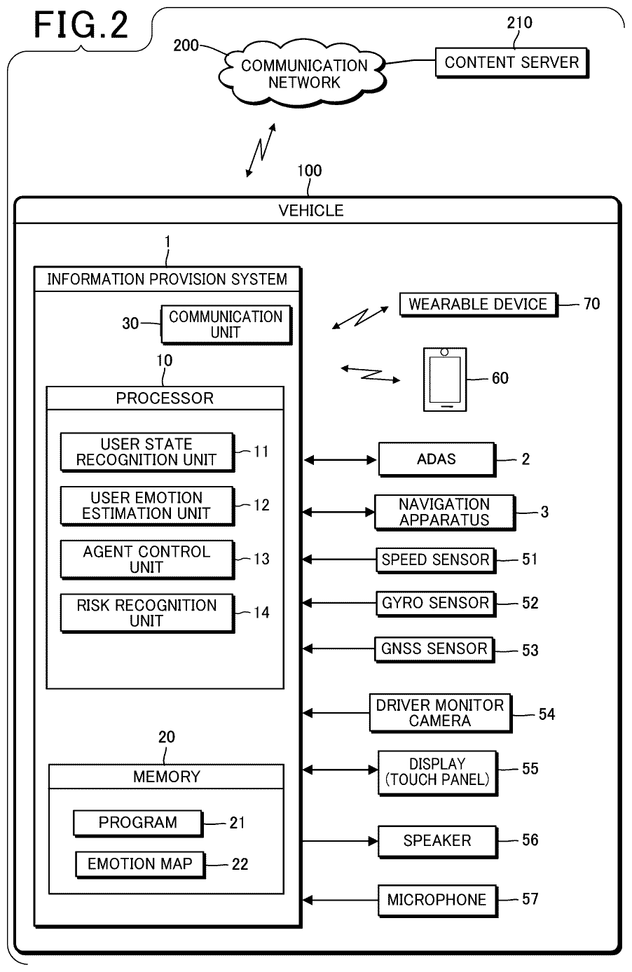

A configuration of the information provision system 1 will be described with reference to FIGS. 2 to 5. With reference to FIG. 2, the information provision system 1 is a control unit including a processor 10, a memory 20, a communication unit 30, and the like. The information provision system 1 is connected to the ADAS 2, a speed sensor 51, a gyro sensor 52, a global navigation satellite system (GNSS) sensor 53, the driver monitor camera 54, the display 55, the speaker 56, and the microphone 57 installed in the vehicle 100.

The communication unit 30 includes a transmitter and a receiver and performs short-range wireless communication between the mobile terminal 60 and the wearable device 70 in accordance with a specification such as Bluetooth (registered trademark) or ultra wideband (UWB). The communication unit 30 performs communication with the content server 210 via the communication network 200.

The memory 20 stores a program 21 for control of the information provision system 1, emotion map 22 data, and the like. The emotion map 22 will be described in detail below. The processor 10 functions as a user state recognition unit 11, a user emotion estimation unit 12, an agent control unit 13, and a risk recognition unit 14, by loading and executing the program 21.

Processing executed by the user state recognition unit 11 corresponds to a user state recognition step in an information provision method of the present disclosure, and processing executed by the user emotion estimation unit 12 corresponds to a user emotion estimation step in the information provision method of the present disclosure. Processing executed by the agent control unit 13 corresponds to an agent control step in the information provision method of the present disclosure, and processing executed by the risk recognition unit 14 corresponds to a risk recognition step in the information provision method of the present disclosure.

The user state recognition unit 11 recognizes the state of the user U based on at least one of information input by a touch operation on the display 55, the image of the user U captured by the driver monitor camera 54, the speech of the user U input to the microphone 57, and the biological information of the user U detected by the wearable device 70, as a state of the user U in the vehicle 100.

The user state recognition unit 11 recognizes, for example, the following elements as the state of the user U.

First element: response to an inquiry “How are you feeling today?” or the like displayed on the display 55 when the user U gets in the vehicle 100, the response being input via a touch operation on the display 55.

Second element: expression and behavior of the user U recognized from the image of the user U.

Third element: utterance content, intonation, pitch, and sound volume of the speech of the user U.

Fourth element: biological information of the user U (heart rate, blood pressure, body temperature, and the like).

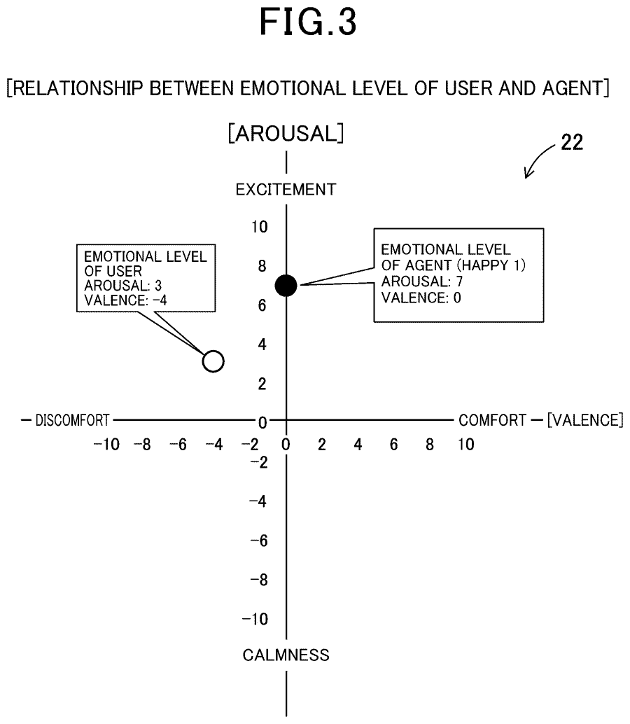

The user emotion estimation unit 12 estimates the emotion of the user U based on the state of the user U recognized by the user state recognition unit 11. The user emotion estimation unit 12 estimates valence and arousal of the user U via the emotion map 22 shown in FIG. 3 as evaluation elements of the emotion. Valence is an evaluation element indicating a level of discomfort/comfort of the user U, and arousal is an evaluation element indicating a level of excitement/calmness of the user U. FIG. 3 shows an example of the levels of the estimated emotion of the user U (arousal: 3, valence: −4) and the set emotional level of the agent (arousal: 7, valence: 0).

The user emotion estimation unit 12 estimates, for example, the emotion of the user U by setting the valence and arousal levels as follows, based on the above first to fourth elements.

Regarding the above first element, the user emotion estimation unit 12 sets the valence of the user U to a relatively high value (for example, 3) when the response to the inquiry is “same as usual,” but sets the arousal to a relatively low value (for example, −3) when the response to the inquiry is “different than usual.”

Regarding the above second element, the user emotion estimation unit 12, based on the expression of the user U, sets the valence to a relatively high value (for example, 5) when it is determined that the user U is showing a smiling expression, but sets the valence to a relatively low value (for example, −5) when it is determined that the user U is showing an annoyed expression. The user emotion estimation unit 12, based on the behavior of the user U, sets the arousal to a relatively high value (for example, 3) when it is determined that the user U is moving restlessly, but sets the arousal to a relatively low value (for example, −3) when it is determined that the user U is hardly moving.

Regarding the above third element, the user emotion estimation unit 12, based on the utterance content of the user U, sets the valence to a relatively high value (for example, 5) when it is determined that the utterance is positive in content such as complimenting something or expecting something positive, but sets the valence to a relatively low value (for example, −5) when it is determined that the utterance is negative in content such as criticizing something. When a specific keyword is included in the content of statements of the user U (such as “good” or “really good”), the user emotion estimation unit 12 sets the valence and arousal values associated with the keyword.

The user emotion estimation unit 12, based on a pitch of the speech of the user U, for example, sets the arousal to a relatively high value (for example, 3) when the pitch of the speech of the user U is higher than or equal to a predetermined pitch, but sets the arousal to a relatively low value (for example, −3) when the pitch of the speech of the user U is lower than the predetermined pitch.

Regarding the fourth element, the user emotion estimation unit 12 applies detected values of the biological information of the user U (heart rate, blood pressure, body temperature, and the like) to a correspondence table of detected values, valence, and arousal prepared in advance, and sets the valence and arousal. The correspondence table may be created based on a profile of the user U input by user U, the biological information of the user U detected in the past, and the like.

The user emotion estimation unit 12 may recognize the valence and arousal values based on a change in a traveling speed of the vehicle 100 detected by the speed sensor 51, a behavior of the vehicle 100 detected by the gyro sensor 52, a variation in a traveling position of the vehicle 100 detected by the GNSS sensor 53, and the like. The user emotion estimation unit 12 may estimate the emotion of the user U based on one of the valence or the arousal, or based on another evaluation element.

The agent control unit 13 provides the user U with the HMI via the agent with anthropomorphic emotions. The agent control unit 13 analyzes the utterance of the user U input to the microphone 57, and outputs a response to the utterance by outputting the speech of the agent from the speaker 56 or through the image display of the agent on the display 55. The agent control unit 13 changes the emotion of the agent depending on the emotion of the user U estimated by the user emotion estimation unit 12, and modifies the output mode of the speech of the agent output from the speaker 56 (intonation, pitch, sound volume, and the like) according to the emotion of the agent. The processing by the agent control unit 13 will be described in detail below.

The risk recognition unit 14 recognizes the predetermined risk faced by the vehicle 100 based on risk information input from the ADAS 2. An example of the predetermined risk faced by the vehicle 100 includes, for example, an approaching monitoring target (other vehicle, pedestrian, obstacle, or the like) present in a vicinity of the vehicle 100. As a distance between the vehicle 100 and the monitoring target decreases, a degree of the risk recognized by the risk recognition unit 14 increases. Note that the risk recognition unit 14 may recognize a degree of concentration of the user U on driving from a facial image of the user U captured by the driver monitor camera 54, the biological information of the user U detected by the wearable device 70, and the like, and may recognize a state in which the degree of concentration has decreased to or below a predetermined level as the predetermined risk.

### 3. Emotion Control Processing of Agent

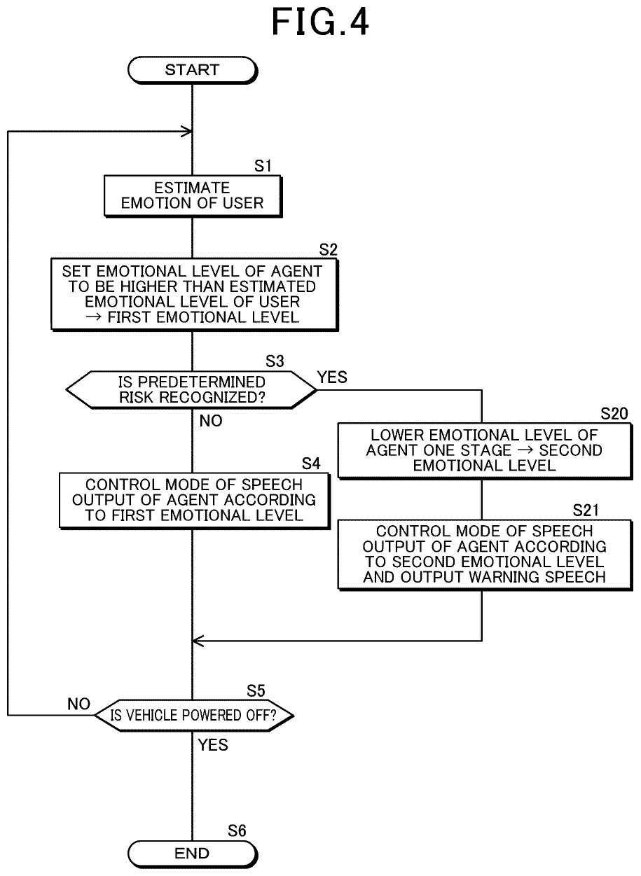

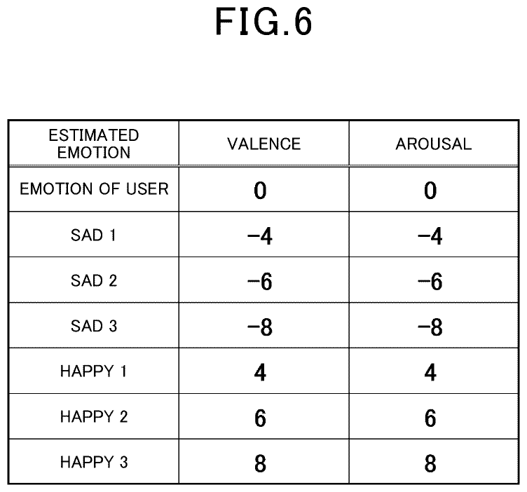

A procedure of emotion control processing of the HMI agent executed by the information provision system 1 according to a flowchart shown in FIG. 4 will be described. When it is recognized that the user U gets in and powers on the vehicle 100, the information provision system 1 starts the processing according to the flowchart shown in FIG. 6.

In step S1 of FIG. 4, the user emotion estimation unit 12 estimates the emotion of the user U based on the state of the user U recognized by the user state recognition unit 11. Next, in step S2, the agent control unit 13 sets the emotional level of the agent higher than the emotional level of the user U with reference to the emotional level of the user U estimated by the user emotion estimation unit 12 in step S1. Note that a high emotional level indicates a state in which both valence and arousal are high.

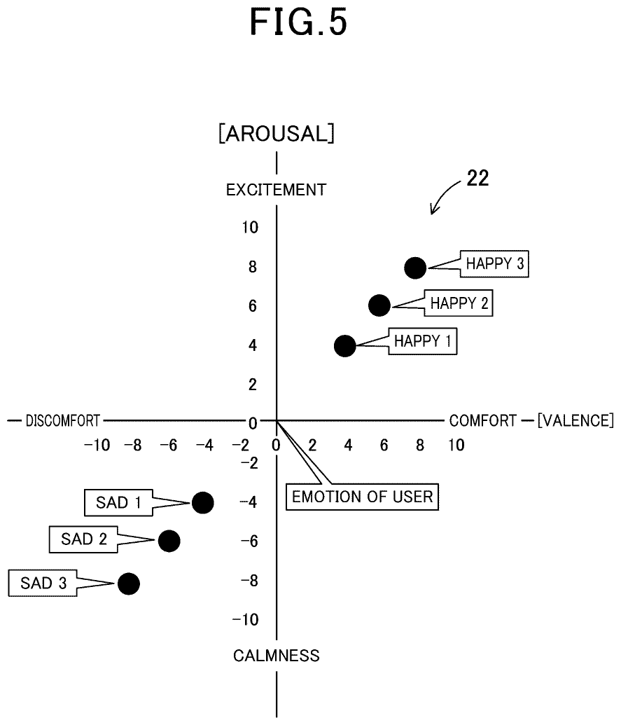

Here, as shown in FIGS. 5 and 6, the agent control unit 13 sets the emotional level of the agent using an emotional level of the user U of valence: 0 and arousal: 0 as starting point, according to any of relative levels (first emotional level) among happy 1, happy 2, and happy 3 when expressing the emotional level in seven stages, namely sad 3, sad 2, sad 1, user emotion, happy 1, happy 2, and happy 3.

Next, in step S3, the agent control unit 13 determines whether the predetermined risk is recognized by the risk recognition unit 14. Then, the agent control unit 13 proceeds the processing to step S20 when the predetermined risk is recognized by the risk recognition unit 14, and proceeds the processing to step S4 when the predetermined risk is not recognized by the risk recognition unit 14.

In step S4, the agent control unit 13 sets and controls the output mode of the speech output from the speaker 56 according to the first emotional level, when the performing speech guidance such as route guidance and content suggestion via the agent, and proceeds the processing to step S5.

In step S20, the agent control unit 13 modifies the emotional level of the agent to a second emotional level being one stage lower than the first emotional level. Next, in step S21, the agent control unit 13 sets and controls the output mode of the speech output from the speaker 56 according to the second emotional level, when performing warning speech output for the predetermined risk via the agent, and proceeds the processing to step S5.

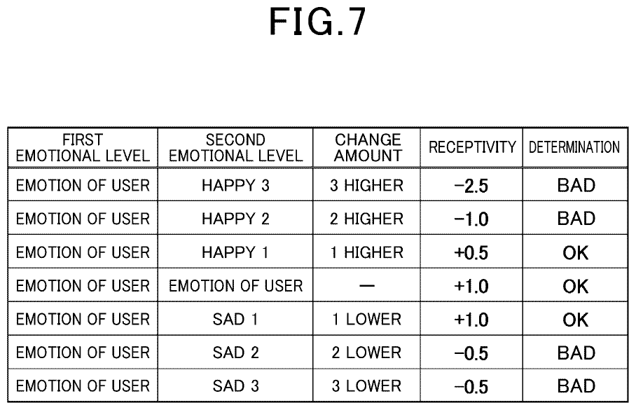

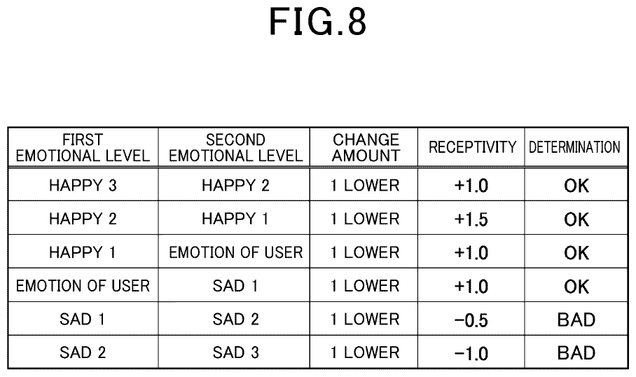

Here, effects of changing the emotional level of the agent and outputting the speech of warning is described with reference to FIGS. 7 and 8. FIG. 7 shows, for a plurality of subjects, evaluation data obtained by monitoring a degree of receptivity (unease) experienced by the subjects, when the emotional level of the agent is changed from an estimated emotional level of the subjects and the warning speech is output.

FIG. 8 shows, for the plurality of subjects, evaluation data obtained by monitoring the degree of receptivity (unease) experienced by the subjects, when the emotional level of the agent is changed from a set emotional level (first emotional level) based on the estimated emotional level of the subjects to the second emotional level and the warning speech is output. FIGS. 7 and 8 show data obtained by evaluating, out of ±3 points, the receptivity (unease) experienced by the subjects, when the emotional level of the agent is changed and the warning speech is output.

Judging from FIG. 7, it can be determined that the receptivity decreases when the change in the emotional level of the agent from the first emotional level to the second emotional level is two levels (two stages) or more. Judging from FIG. 8, it can be determined that the receptivity can be further increased, by setting the first emotional level higher than the emotional level of the subjects and decreasing the second emotional level to be one level lower (1 lower) than the first emotional level. In particular, when the first emotional level is set to happy 2 being two levels higher than the emotion of the user and the second emotional level is set to happy 1 being one level lower than happy 2, the receptivity is at its highest at +1.5.

Based on this evaluation result, the agent control unit 13 sets the first emotional level of the agent to be higher than the emotional level of the user U in step S2 of FIG. 4, and sets the second emotional level of the agent to be one level lower than the first emotional level in step S20. With this, it is possible to enhance the effect of the warning while reducing output of the warning speech by the agent and any unease experienced by the user U.

In step S5 of FIG. 4, the agent control unit 13 determines whether the vehicle 100 has been powered off. When the vehicle 100 has not been powered off, the agent control unit 13 then proceeds the processing to step S1 and executes the processing in steps S1 to S5, S20, S21 again. On the other hand, when the vehicle 100 has been powered off, the agent control unit 13 proceeds the processing to step S6 and ends the emotion control processing of the agent.

### 4. Other Embodiments

In the above embodiment, the vehicle 100 is shown as the mobile body of the present disclosure, but the mobile body of the present disclosure may be an aircraft, a ship, or the like as long as the vehicle 100 is a mobile body that provides the HMI using the agent to the user in the vehicle 100.

In the above embodiment, the information provision system of the present disclosure is exemplified as the information provision system 1 configured as an in-vehicle device installed in the vehicle 100. As another embodiment, part or all of the information provision system of the present disclosure may be configured as the mobile terminal 60 brought into the vehicle 100, or as an external communication system such as the content server 210 outside of the vehicle 100.

When configuring the information provision system as the mobile terminal 60, it is possible to provide the HMI utilizing the agent by using a camera, a microphone, a speaker, and a display included in the mobile terminal 60. The biological information of the user U may be obtained through communication between the wearable device 70 and the mobile terminal 60, and the emotion of the user U may be estimated by the mobile terminal 60 based on the biological information. The mobile terminal 60 may acquire information about the predetermined risk recognized by the ADAS 2 through communication between the in-vehicle device of the vehicle 100 and the mobile terminal 60.

When configuring the information provision system as an external communication system, for example, the image of the user U captured by the driver monitor camera 54 of the vehicle 100, speech data input to the microphone 57, and the like, as well as the information about the predetermined risk recognized by the ADAS 2 are transmitted from the vehicle 100 to the external communication system. This configuration allows the external communication system to set the emotion of the agent.

In the above embodiment, the user state recognition unit 11 and the user emotion estimation unit 12 are provided, and the agent control unit 13 sets the emotion of the agent based on an estimated value of the emotion of the user U. As another configuration, the user state recognition unit 11 and the user emotion estimation unit 12 may be omitted, and the emotion of the agent in a regular state in which the predetermined risk is not recognized by the ADAS 2 may be set in advance to a predetermined level (the first emotional level of the present disclosure). In this case, when switching from a state in which the predetermined risk is not recognized by the ADAS 2 to a state in which the predetermined risk is recognized by the ADAS 2, the agent control unit 13 is configured to modify the emotional level of the agent to an emotional level with a lower degree of happiness (corresponding to the second emotional level of the present disclosure). Note that the emotional level of the agent in the regular state may be set based on a profile or the like of the user U.

Note that FIG. 2 is a schematic diagram showing the configuration of the information provision system 1 by dividing the configuration according to a main processing content to facilitate understanding of the invention of the present application, and the configuration of the information provision system 1 may be divided differently. The processing of respective components may be executed by one hardware unit or may be executed by a plurality of hardware units. The processing of each component shown in FIG. 4 may be executed by one program or may be executed by a plurality of programs.

### 5. Configurations Supported by the Above Embodiments

The above embodiments are specific examples of the following configurations.

### Configuration 1

An information provision system including: a risk recognition unit that recognizes a predetermined risk faced by a mobile body; and an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.

According to the information provision system of Configuration 1, it is possible to reduce causing the user to feel uneasy, when issuing a warning via the HMI using the agent to the user riding the vehicle.

### Configuration 2

The information provision system according to Configuration 1, further including: a user state recognition unit that recognizes a state of the user; and a user emotion estimation unit that estimates an emotion of the user, based on a recognition result of the state of the user by the user state recognition unit, wherein the agent control unit sets the emotion of the agent based on the emotion of the user estimated by the user emotion estimation unit, when the predetermined risk is not recognized by the risk recognition unit.

According to the information provision system of Configuration 2, it is possible to enhance the effect of the warning when the predetermined risk has occurred while achieving natural interaction between the user and the agent, by setting the emotion of the agent based on the emotion of the user.

### Configuration 3

The information provision system according to Configuration 2, wherein the agent control unit sets the emotion of the agent to an emotional level having a higher degree of happiness than that of an emotional level of the user estimated by the user emotion estimation unit, when the predetermined risk is not recognized by the risk recognition unit.

According to the information provision system of Configuration 3, the first emotional level of the agent in Configuration 1 set by the agent control unit becomes the emotional level having a higher degree of happiness than the emotional level of the user estimated by the user emotion estimation unit, thereby making it possible to enhance the effect of reducing any unease the user experiences when issuing a warning to the user.

### Configuration 4

The information provision system according to Configuration 2 or 3, wherein the user emotion estimation unit uses valence as an estimation element of the emotion of the user, and the agent control unit uses valence as a control element of the emotion of the agent.

According to the information provision system of Configuration 4, it is possible to estimate the emotion of the user using the valence being a scale for evaluating comfort and discomfort, and to control the emotion of the agent.

### Configuration 5

The information provision system according to any one of Configurations 2 to 4, wherein the user emotion estimation unit uses arousal as an estimation element of the emotion of the user, and the agent control unit uses arousal as a control element of the emotion of the agent.

According to the information provision system of Configuration 5, it is possible to estimate the emotion of the user using the arousal being a scale for evaluating excitement and calmness, and to control the emotion of the agent.

### Configuration 6

An information provision method executed by a computer including: a risk recognition step of recognizing a predetermined risk faced by a mobile body; and an agent control step of providing, using an agent having an anthropomorphic emotion, a user in the mobile body with a human machine interface (HMI) that performs speech output in a mode corresponding to the emotion of the agent, and outputting warning speech via the agent when the predetermined risk is recognized in the risk recognition step, wherein in the agent control step: the emotion of the agent is set according to an emotional level associated with happiness and sadness; and when the emotion of the agent is set to a first emotional level, the emotion of the agent is modified to a second emotional level having a degree of happiness lower than that of the first emotional level and the warning speech is output via the agent, when switching from a state in which the predetermined risk is not recognized in the risk recognition step to a state in which the predetermined risk is recognized in the risk recognition step.

It is possible to achieve advantageous effects similar to those of the information provision system of Configuration 1 through the computer executing the information provision method of Configuration 6.

### Configuration 7

A non-transitory computer-readable storage medium storing a program for causes a computer to function as: a risk recognition unit that recognizes a predetermined risk faced by a mobile body; and an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.

It is possible to achieve the configuration of the information provision system of Configuration 1 through the computer executing the program of Configuration 7.

### REFERENCE SIGNS LIST

1: information provision system
2: ADAS
10: processor
11: user state recognition unit
12: user emotion estimation unit
13: agent control unit
14: risk recognition unit
20: memory
21: program
22: emotion map
30: communication unit
50: front camera
51: speed sensor
52: gyro sensor
53: GNSS sensor
54: driver monitor camera
55: display
56: speaker
57: microphone
60: mobile terminal
70: wearable device
100: vehicle (mobile body)
200: communication network
210: content server
U: user

1. An information provision system comprising:
a risk recognition unit that recognizes a predetermined risk faced by a mobile body; and
an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.

2. The information provision system according to claim 1, further comprising:
a user state recognition unit that recognizes a state of the user; and
a user emotion estimation unit that estimates an emotion of the user, based on a recognition result of the state of the user by the user state recognition unit, wherein
the agent control unit sets the emotion of the agent based on the emotion of the user estimated by the user emotion estimation unit, when the predetermined risk is not recognized by the risk recognition unit.

3. The information provision system according to claim 2, wherein the agent control unit sets the emotion of the agent to an emotional level having a higher degree of happiness than that of an emotional level of the user estimated by the user emotion estimation unit, when the predetermined risk is not recognized by the risk recognition unit.

4. The information provision system according to claim 2, wherein
the user emotion estimation unit uses valence as an estimation element of the emotion of the user, and
the agent control unit uses valence as a control element of the emotion of the agent.

5. The information provision system according to claim 2, wherein
the user emotion estimation unit uses arousal as an estimation element of the emotion of the user, and
the agent control unit uses arousal as a control element of the emotion of the agent.

6. An information provision method executed by a computer, the information provision method comprising:
a risk recognition step of recognizing a predetermined risk faced by a mobile body; and
an agent control step of providing, using an agent having an anthropomorphic emotion, a user in the mobile body with a human machine interface (HMI) that performs speech output in a mode corresponding to the emotion of the agent, and outputting warning speech via the agent when the predetermined risk is recognized in the risk recognition step, wherein
in the agent control step: the emotion of the agent is set according to an emotional level associated with happiness and sadness; and when the emotion of the agent is set to a first emotional level, the emotion of the agent is modified to a second emotional level having a degree of happiness lower than that of the first emotional level and the warning speech is output via the agent, when switching from a state in which the predetermined risk is not recognized in the risk recognition step to a state in which the predetermined risk is recognized in the risk recognition step.

7. A non-transitory computer-readable storage medium storing a program for causing a computer to function as:
a risk recognition unit that recognizes a predetermined risk faced by a mobile body; and
an agent control unit that provides, using an agent having an anthropomorphic emotion, a user in the mobile body with a human machine interface (HMI) that performs speech output in a mode corresponding to the emotion of the agent, and outputs warning speech via the agent when the predetermined risk is recognized by the risk recognition unit, wherein
the agent control unit: sets the emotion of the agent according to an emotional level associated with happiness and sadness; and when the emotion of the agent is set to a first emotional level, modifies the emotion of the agent to a second emotional level having a degree of happiness lower than that of the first emotional level and outputs the warning speech via the agent, when switching from a state in which the predetermined risk is not recognized by the risk recognition unit to a state in which the predetermined risk is recognized by the risk recognition unit.
